(19) United States
(12) Patent Application Publication
(10) Pub. No.: US 2015/0148630 A1
(43) Pub. Date: May 28, 2015

(54) METHOD AND DEVICE FOR DETECTING FLUORESCENCE RADIATION

(71) Applicant: QUEST PHOTONIC DEVICES B.V., Middenmeer (NL)

(72) Inventor: Richard Johannes Cornelis Meester, Middenmeer (NL)

(73) Assignee: QUEST PHOTONIC DEVICES B.V., Middenmeer (NL)

(21) Appl. No.: 14/412,694

(22) PCT Filed: Jul. 4, 2013
(86) PCT No.: PCT/NL2013/050493

§ 371 Date: Jan. 5, 2015

(30) Foreign Application Priority Data
Jul. 5, 2012 (NL) 2009124

(51) Int. Cl. A61B 5/00; A61B 1/00; A61K 49/00; G01N 21/64; A61B 1/04

(52) U.S. Cl. CPC A61B 5/0071; G01N 21/6456; A61B 5/0084; A61B 5/7425; A61B 1/043; A61K 49/0017; A61B 1/00193; G01N 2201/08; A61B 2576/02
USPC 600/317

(57) ABSTRACT

A method for detecting fluorescence radiation from a fluorescence agent, includes:
emitting light at an excitation wavelength range (72) to cause fluorescence radiation emission in the fluorescence agent, the fluorescence radiation having a fluorescence wavelength profile (73);
detecting light at a first fluorescence wavelength range (74) as a first detection signal (S1);
detecting light at a second fluorescence wavelength range (81, 91) as a second detection signal (S2); and
numerically determining a third detection signal with an improved fluorescence-to-background radiation ratio based on the first detection signal (S1), the second detection signal (S2), and the fluorescence wavelength profile (73).

### FIELD OF THE INVENTION

The invention relates to a method for detecting fluorescence radiation from a fluorescence agent using a probe such as an endoscope tip, to an endoscope tip suitable to perform said method, and to an endoscope system configured to perform said method. The invention also relates to an optical system comprising a camera and lens forming a probe other than an endoscope.

### BACKGROUND OF THE INVENTION

In fluorescence imaging applications, a fluorescence dye or other fluorescence substance is applied as a labelling agent in an (internal) body part. With light at a specific wavelength (the excitation wavelength) from a light source such as a laser or LED, the fluorescence agent is excited. As a result, fluorescence light at a secondary wavelength is emitted by the agent. This light is sampled by an imaging sensor, such as a CCD sensor, of a probe to obtain a fluorescence signal. Especially when the probe must detect the fluorescence light through skin and tissue, the signal to noise ratio can be low. High gain usually needs to be applied to get a suitable signal level. In addition, scattering of fluorescence photons in tissue further reduces the signal to noise ratio.

This effect is usually overcome by using long integration times to increase the number of photons reaching the imaging sensor and therefore increasing the fluorescence signal and the signal to noise ratio.

Besides fluorescence radiation, the sensor also picks up so-called background radiation that is not caused by the excited fluorescence agent. Since the aim of fluorescence imaging is to view only the light emitted from the fluorescence agent, this background radiation should be separated from the measured fluorescence signal.

In some cases, the background signal is suppressed by applying a threshold criterion to the sensor signal. In real time systems during surgery however with varying light conditions this is no viable solution. The threshold level is varying and hence the background signal can be higher than the threshold level, rendering the threshold useless, or the total background and fluorescence signal can be lower than the threshold, removing both the fluorescence and background signal.

US 2002/0062061 discloses a method and system for detecting fluorescence radiation intensities at two different wavelength ranges. Moving parts and filters are used to measure at different wavelengths sequentially, not simultaneously. The ratio of the two intensities is used to determine whether or not tissue is healthy. There is no correction for any background radiation at the fluorescence wavelengths. U.S. Pat. No. 7,722,534 also discloses a method and system for detecting fluorescence in which tissue suspicious for early cancer is identified based on the ratio between a fluorescence signal and a reference signal.

The cited prior art thus primarily addresses the problem of measuring and classifying a fluorescence spectrum. It does not address the problem of separating measured fluorescence radiation from background radiation.

### OBJECT OF THE INVENTION

It is an object of the invention to provide a method and device for fluorescence imaging that overcomes at least one of the mentioned drawbacks.

### SUMMARY OF THE INVENTION

The invention provides a method for detecting fluorescence radiation from a fluorescence agent, the method comprising
emitting light at an excitation wavelength range for causing fluorescence radiation emission in the fluorescence agent, said fluorescence radiation having a fluorescence wavelength profile;
detecting light at a first fluorescence wavelength range as a first detection signal;
detecting light at a second fluorescence wavelength range as a second detection signal;
numerically determining a third detection signal with an improved fluorescence-to-background radiation ratio based on the first detection signal, the second detection signal, and the fluorescence wavelength profile.

By measuring at two different fluorescence wavelength ranges, and using knowledge of the fluorescence emission distribution curve at least in those ranges, the influence from the background radiation to the measured signal can be numerically reduced or practically eliminated. For example, based on the knowledge of the fluorescence curve, it is known what the ratio of the intensities at the two wavelength ranges should be in the absence of background radiation. In the presence of background radiation, which has an assumedly uniform intensity distribution at the two wavelengths, this ratio is modified. Using the knowledge of the unmodified ratio, it becomes possible to separate the contribution from the fluorescence radiation from the contribution from the background radiation. Thus, the signal to noise (fluorescence-to-background) ratio is advantageously improved. In addition, any following classification step of the measured fluorescence radiation may be improved, since the contribution of the background is removed or at least strongly diminished.

In an embodiment according the invention, the measurements at the at least two different fluorescence wavelength ranges are done simultaneously. A simultaneous rather than sequential measurement prevents problems with non-stationary samples. It can also allow for a more robust implementation as sequential measuring techniques often make use of moving parts (e.g. rotating mirrors or filter sets).

In an embodiment according the invention, the method further comprises
generating a fluorescence image based on the third detection signal;
showing said fluorescence image on a display.

In an embodiment according the invention, the method further comprises
detecting visible light as a fourth detection signal;
merging the fluorescence image with an image based on the fourth detection signal.

This way an image is obtained containing both visible details and the fluorescence radiation. The position of the fluorescence agent is thus easier to determine, and the fluorescence image will be easier to interpret.

In an embodiment according the invention, the detected light (fluorescence and/or visible light) is captured via a single incident light entry surface, so that the respective detection signals are spatially aligned.

In an embodiment according the invention, the method comprises applying a numerical criterion to determine if a pixel in a measured fluorescence image contains essentially only background radiation and, if said numerical criterion is satisfied, removing or darkening the pixel in the measured fluorescence image. That way, detected radiation that appears to be fluorescence but is in fact background radiation can be removed from a measured image, so that only the fluorescence radiation remains. The fluorescence radiation is what the operator of the method is typically primarily interested in. The criterion to numerically determine if a pixel comprises essentially only background radiation can be provided in different ways. For example, the criterion may be that the background radiation may comprise no more than 80%, 90%, or 95% of the total measured radiation, as indicated by the action of determining the separation of background radiation and fluorescence radiation.

In an embodiment according the invention, numerically determining the third detection signal comprises calculating the difference of the first detection signal and the second detection signal. In particular, numerically determining the third detection signal can comprise evaluating (S1−S2)/(1−x), wherein S1 represents a detection signal in the first fluorescence range , S2 represents a detection signal in the second fluorescence range, and x is the calculated ratio of light emitted in the first florescence wavelength range and light emitted in the second fluorescence wavelength range according to the fluorescence wavelength profile.

In an embodiment according the invention, the second fluorescence wavelength range is at a wavelength range where the fluorescence wavelength profile has a normalized value of at least 0.2. In an embodiment according the invention, the second fluorescence wavelength range is at a wavelength range where the fluorescence wavelength profile has a normalized value that is less than 0.2.

In an embodiment according the invention, the light at the excitation wavelength is emitted from an endoscope tip, and the detectors are comprised in said endoscope tip. In an alternative embodiment, the light at the excitation wavelength is emitted from a light source external to a fluorescence measuring probe (such as the mentioned endoscope tip). In any system, the light at the first and/or the second fluorescence wavelength ranges may be detected using a prism based camera system. This advantageously allows simultaneous measurements at both fluorescence radiation wavelengths.

The invention also provides a measurement device for measuring fluorescence radiation from a fluorescence agent having a fluorescence wavelength profile, the device comprising
a wavelength separation device configured to receive incident light originating from the agent and to separate said light into a plurality of channels;
at least two imaging sensors connected to at least two respective channels of the plurality of channels, wherein the first channel is configured for transmitting light at a first fluorescence wavelength range, from which the respective sensor will generate a first detection signal, and the second channel is configured for light at a second fluorescence wavelength range, from which the respective sensor will generate a second detection signal;
a processing device configured for numerically determining a third detection signal with an improved fluorescence-to-background radiation ratio based on the first detection signal, the second detection signal, and the fluorescence wavelength profile.

The measurement device according the invention advantageously allows simultaneous measurements at both fluorescence radiation wavelengths. In addition, the design is more compact and robust than prior art systems comprising movable parts for sequential measurements.

In an embodiment according the invention, the measurement device is configured for use as an endoscope tip, wherein the wavelength separation device is a dichroic prism assembly. The device may be further provided with fibers for transmitting excitation light to excite the fluorescence agent. The dichroic prism assembly can have at least three channels, the third channel being configured for transmitting light at a visible wavelength range, from which the respective sensor can generate a fourth signal representative of the visible environment of the endoscope tip.

The invention further provides an endoscope system comprising an endoscope tip as described above, and processing means for numerically determining a third detection signal with an improved fluorescence-to-background radiation ratio based on the first detection signal, the second detection signal, and the fluorescence wavelength profile, as also described above.

The invention further provides a probe system comprising a fluorescence measurement device as described above, such as an open system fluorescence measurement device, and processing means for numerically determining a third detection signal with an improved fluorescence-to-background radiation ratio based on the first detection signal, the second detection signal, and the fluorescence wavelength profile.

### BRIEF DESCRIPTION OF THE FIGURES

On the attached drawing sheets,

FIG. 1 schematically shows light paths through a dichroic prism assembly;

FIG. 2 schematically shows a perspective view of an extended dichroic prism assembly module according to an embodiment of the invention;

FIG. 3 schematically shows a perspective view dichroic prism assembly for use in a fluorescence probe according to an embodiment of the invention;

FIGS. 4 and 5 schematically show cross sections of an endoscope tube comprising a dichroic prism assembly according to an embodiment of the invention;

FIG. 6 schematically shows a perspective view of an endoscope tube according to an embodiment of the invention with part of the tube wall removed;

FIG. 7 shows an excitation and fluorescence wavelength distribution;

FIGS. 8 and 9 show excitation and fluorescence wavelength distributions and light sampling wavelength ranges according to an embodiment of the invention;

FIGS. 10, 11, and 12 show further fluorescence wavelength distributions and light sampling wavelength ranges according to an embodiment of the invention; and

FIG. 13 schematically shows a fluorescence measurement probe according to an embodiment of the invention.

### DETAILED DESCRIPTION

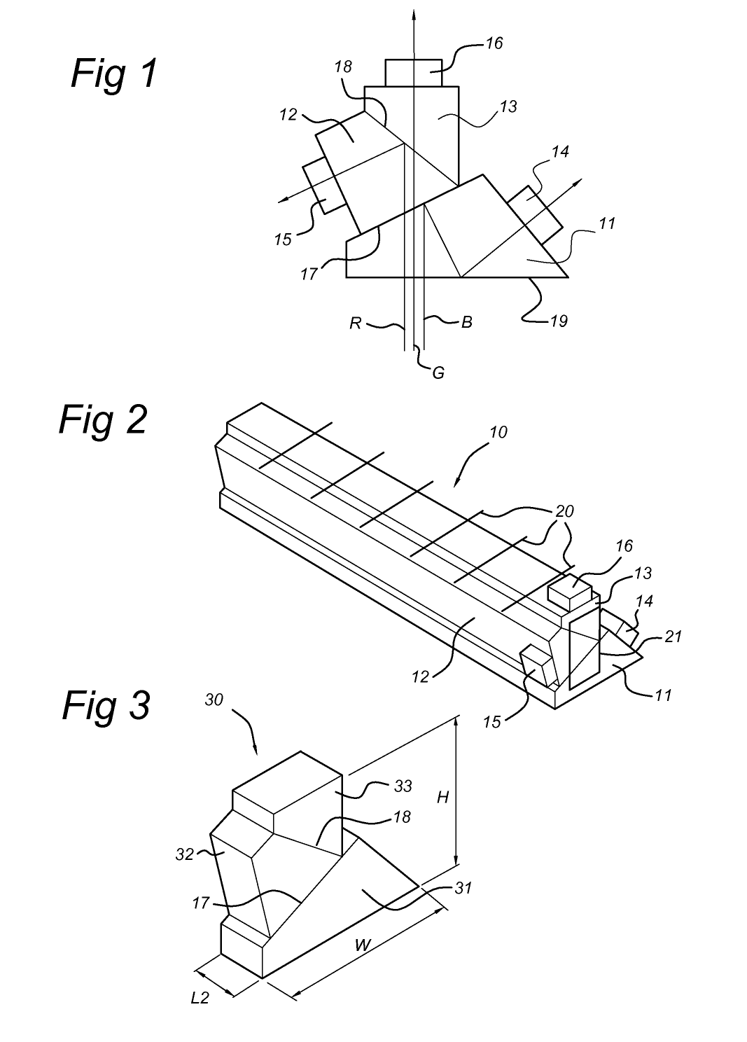

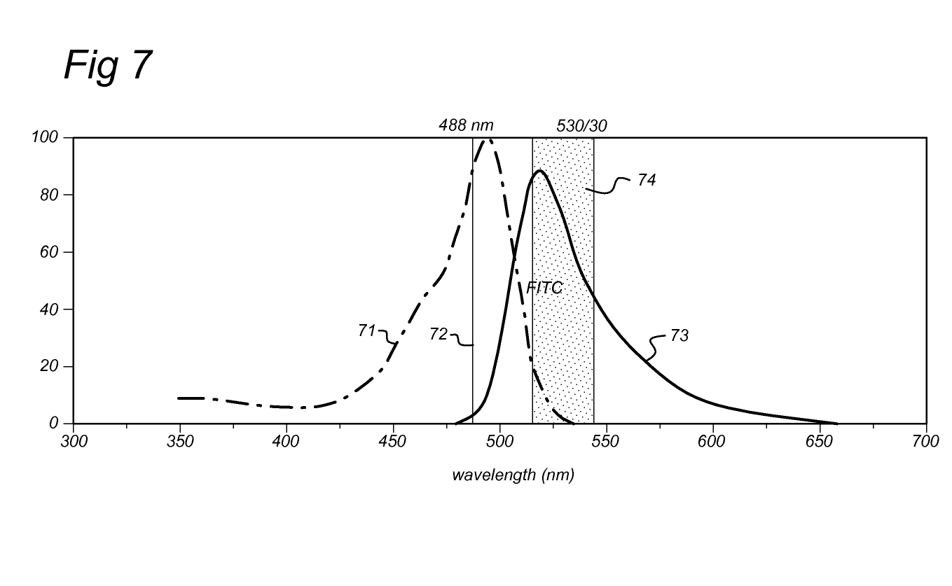

FIG. 1 schematically shows light paths through a dichroic prism assembly. An exemplary dichroic prism assembly configured to separate light into red R, green G, and blue B components will now be discussed to illustrate the functioning of such assembly. However, the invention is not limited to separation into R, G, and B. In reference to FIGS. 7-12, other wavelengths will be discussed. It will be clear to a skilled person that a dichroic prism assembly is a light separation means which can be configured to separate light into arbitrary wavelengths.

Returning to the exemplary assembly of FIG. 1, light comprising red R, green G and blue B components enters the assembly through incident surface 19, shown here as the bottom surface of the assembly. The first transition surface 17, between the first 11 and second prisms 12 comprises a coating that is configured to reflect blue light and transmit red and green light. The blue component B is nearly totally reflected and, due to the shape of first prism 11, exits the first prism through the side where sensor 14 is attached. The applied coating can be a grated refraction index coating.

The green G and red R components pass through the first transition surface 17. The second transition surface 18, between the second 12 and third 13 prisms, is provided with a coating, for example another grated refraction index coating, that reflects red light but allows green light to pass. The red light is thus reflected at surface 18 and exits the second prism through the face on which the second sensor 15 is attached. The green light passes through second transition surface 18 and third prism 13 and exits through the face on which third sensor 16 is attached. Each of these paths through the prism assembly is known as a channel.

It is again noted that the invention is not limited to the exemplary R, G, and B separation. Any configuration of components can be used, as determined by the reflection/transmission wavelength of the coating(s) used. For example, suitable coatings may be used that so that one channel includes light in the wavelength range of 400 to 650 nm (blue, green, and red), another light in the range 650 to 750 nm (red, near-infrared) and a third channel has light in the range 750 to 1000 nm (infrared). In addition, filters may be placed between the exit of the prism and the sensor.

Returning to the example of FIG. 1, the red, green, and blue, R, G, B, components are thus sampled by first, second and third detectors 14, 15, and 16. As mentioned before, these principles apply to any light components, not necessarily red, green and blue, provided that suitable coatings of surfaces 17 and 18 and material for prisms 11, 12, 13 is used.

Conventionally, air gaps are often used to provide a second transient surface 17 suitable for reflecting red light. According to the invention, also a grated refraction index coating may be used on any transient surface 17. Such a coating can be in principle applied for any wavelength. Such a coating removes the need for air gaps, which is advantageous since air gaps may be filled with dust when the module is cut.

FIG. 2 schematically shows a perspective view of an dichroic prism assembly module 10, comprising three extended prisms 11, 12, 13. Vacuum bonding is performed by pressing the small uncut pieces together. In order to further fortify the bonding, a glass sheet 21 is attached to each side of the module (front and back). This sheet may later be removed, when the formed dichroic prism assembly for use in an endoscope is formed. The sheet can also remain in the formed dichroic prism assembly.

According to an embodiment of the invention, the dichroic prism assembly module 10, having at least one dimension unsuitable for use in an endoscope tip is cut along a cutting line 20. FIG. 2 shows several examples of cutting lines 20. After cutting, at least one dichroic prism assembly 30 suitable for use in an endoscope tip is obtained. Repeated cuttings will yield a plurality of dichroic prism assemblies 30.

FIG. 3 shows an example of an dichroic prism assembly 30 obtained by the described cutting process. The assembly 30 has width W, height H, and length L₂. Length L₂ is much smaller than the length L of the module 10 of which assembly 30 was a part. A typical value for L₂ is between 0.5 mm and 2 mm. Typical values for H are between 0.5 mm and 2 mm, and for W also between 0.5 mm and 2 mm.

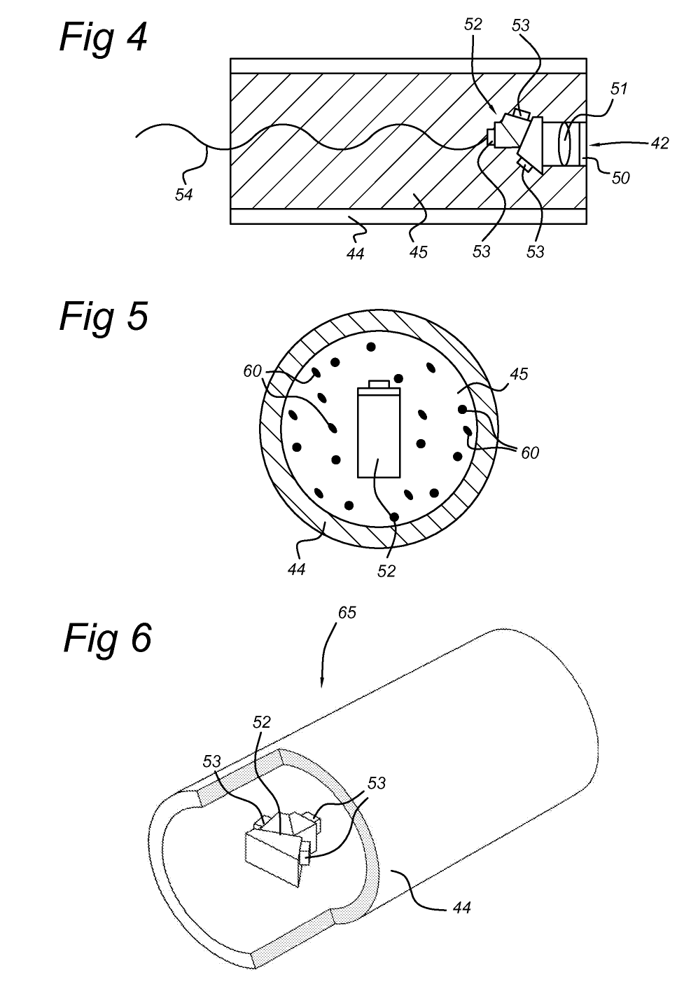

In FIG. 4, a length-wise cross section of an endoscope tip according an embodiment of the invention is shown. The incident light that enters the endoscope tip along incident path 42 is transmitted through cover plate 50, focused by a lens 51 onto a dichroic prism assembly 52 according the invention. The assembly 52 may be obtained by the above described method of cutting a module 10. The assembly 52 is dimensioned to be suitable for use in an endoscope tip. The dimensions of the assembly 52 may be between 0.5 and 5 mm in each direction, preferably between 0.5 and 2 mm or between 1 and 1.5 mm.

The dichroic prism assembly 52 is provided with sensors 53. These sensors may comprise Charge-Coupled Devices (CCDs). The sensors may also comprise a chip comprising means for determining a relative or absolute orientation, or rate of change of said orientation, of the endoscope tip. An example is a so-called gyro chip. The endoscope tip may also comprise processing means, for example for processing pixel data from the CCD. Connected to the sensors are signal wires 54 for carrying a signal from the sensor and/or chip in the sensor away from the endoscope tip, typically to an external signal processing device such as a PC or monitoring device.

In FIG. 5, a cross section of tube wall 44 is shown. The interior 45 comprises optical fibers 60 or bundles of fibers 60. These fibers may be used to transport light from an external light source, through the transparent front surface 45 to illuminate an area surrounding the endoscope tip. The reflecting light is then received via the first and second incident paths 42 and 43. Because two incident light paths are provided, the endoscope can be used for stereo imaging.

FIG. 6 schematically shows a perspective view of an endoscope tube according the invention with part of the tube wall 44 removed, and without the fibers 60, tense 51 and cover surfaces 45 and 50.

The endoscopes according the invention are, however, not limited to endoscope tips with one incident paths 42 as shown in FIGS. 4, 5 and 6. Endoscopes with two (e.g. for stereo applications) or three or more incident paths can also be envisaged. Not all paths need to be provided with a dichroic prism assembly according the invention—only where the light needs to be separated into several components.

FIG. 7 shows excitation 71 and emission 73 curves for Fluorescein Isothiocyanate (FITC). Many other fluorescence agents are available, such as Indocyanine Green (ICG), CW-800, Cy5, Cy5.5, etc., each with their respective excitation and emission curves. The x-axis shows the wavelength (in nanometres, nm) of the excitation or emission wavelength. FITC has a peak excitation wavelength of approximately 495 nm, and a peak fluorescence emission wavelength of approximately 521 nm. As excitation source typically a laser, LED, or other light source having a narrow emission profile 72 close to the peak excitation wavelength is used. In the present example, nearly monochromatic laser light at 488 nm is used as excitation source.

To measure the fluorescence, typically a narrow band filter is placed in the optical path of the detector to only sample the emission wavelength close to the top of emission, but away from the excitation wavelength. Furthermore the excitation source wavelength is blocked from reaching the sensor. In the present example, a filter having a bandwidth of approximately 30 nm is used around a central wavelength of approximately 530 nm.

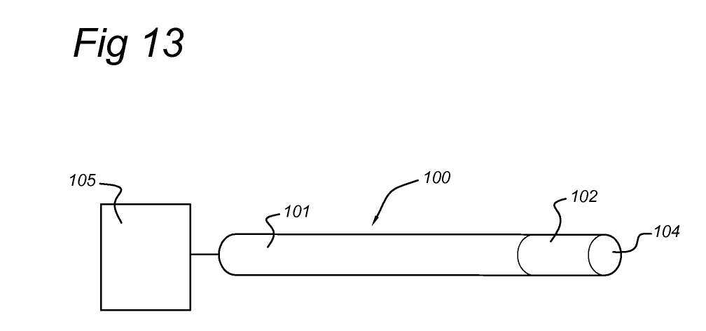

In fluorescence endoscopy applications using an endoscope having an endoscope tip as shown in FIGS. 4, 5, and 6, at least one but typically more fibers 60 emit light at the excitation wavelength. Other fibers may emit light in the visible range (e.g. white light), so that the endoscope can also register a visible image, for example to aid the operator of the endoscope in navigating. In case of open systems (see e.g. FIG. 13) the excitation wavelength and visible light can be supplied by any general illumination apparatus.

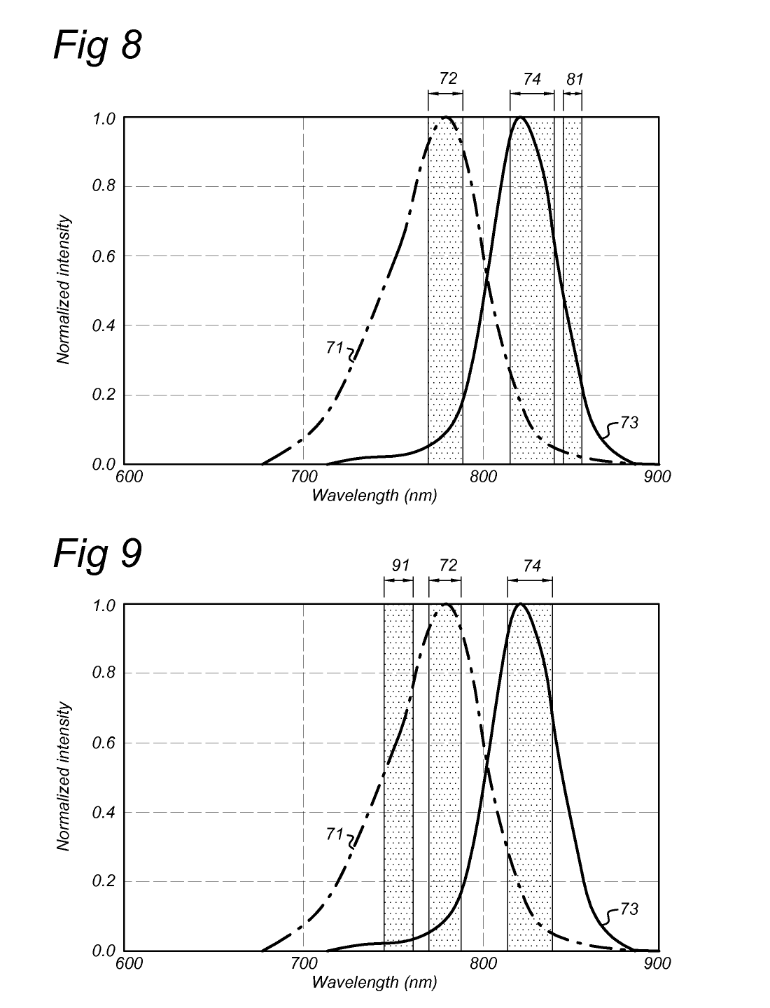

In an embodiment according the invention the endoscope tip is provided with a dichroic prism assembly 52 configured to split light into three wavelength ranges and provided with a respective sensor 14, 15, 16 for each of the three wavelength ranges. A first wavelength range may be a first fluorescence wavelength range. The second wavelength range may be a second fluorescence wavelength range (preferably not overlapping the first wavelength range, in any case not identical to the first wavelength range) and the third wavelength range may be in the visible light range. As was mentioned before, by sensing the visible light in one channel, the endoscope can transmit a gray-scale image that may aid the operator of the endoscope. The use of the first and second fluorescence wavelength ranges will be discussed in reference to FIGS. 8 and 9.

FIG. 8 shows an example of an excitation wavelength range 72 (near the peak of the exemplary excitation curve 71), a first fluorescence wavelength range 74 near the peak of the fluorescence curve 73, and a second fluorescence wavelength range 81 In the example of FIG. 8, the first fluorescence wavelength range 74 overlaps with the emission curve near the peak value. That is, the normalized (i.e. the peak value corresponds to 1.0) emission intensity of the overlapped part of the fluorescence emission profile is between 0.6 and 1.0. The first fluorescence wavelength range is thus close to the peak of the emission profile 73 and may overlap with the peak wavelength, as is the case in FIG. 8. Other ranges 74 are also possible, for example overlapping parts of the emission curve 73 where the normalized intensity is between 0.4 and 1.0, between 0.5 and 1.0, and between 0.8 and 1.0.

In the example of FIG. 8, the second fluorescence wavelength range 81 overlaps with the emission curve 73 in an area where the normalized emission intensity is between 0.2 and 0.6. Other ranges 81 are also possible, for example overlapping parts of the emission curve 73 where the normalized intensity is between 0.2 and 0.4, between 0.2 and 0.6, between 0.2 and 0.8 and between 0.2 and 1.0.

In an embodiment according the invention, the first wavelength range 74 is closer to the peak emission wavelength than the second wavelength range 81.

Let S1 denote the signal detected by the sensor detecting light of the first wavelength range 74 and S2 denote the signal detected by the sensor detecting light of the second wavelength range 81 can be calculated. In an approximation, the background emission B is independent of the wavelength. The detectors will thus detect a combination of a wavelength independent background emission B and wavelength dependent fluorescence radiation P1 (averaged over the first wavelength range) and P2 (averaged over the second wavelength range). In formula form: S1=B+P1 and S2=B+P2. In these formulas, the emissions are presented per unit of wavelength interval, to account for the differences in wavelength range widths.

Since the fluorescence emission curve 73 is known, the number of variables (B, P1, and P2) can be reduced from three to two. Based on the knowledge of the emission curve 73, P2 can be expressed as a fraction of P1 (considering that the first wavelength range is closer to the peak emission wavelength than the second range, so that P1>P2), i.e. P2=×P1, where x is a real number between 0 and 1.

Now the background radiation contribution B can be eliminated from the equations, to obtain for example the following expression for P1: P1=(S1−S2)/(1−x). Thus, a third detection signal is obtained from which the background radiation is largely eliminated. in a further embodiment, the third detection signal is calibrated using known procedures so that a quantitative fluorescence measurement is obtained.

In FIG. 9, the second wavelength range 91 is chosen at a larger distance from the first wavelength range so that it can be said to overlap the tail of the emission distribution. It follows that the range 91 overlaps with a part of the emission curve having lower normalized intensity values, i.e. between 0.0 and 0.1. Other exemplary overlap ranges are between normalized intensity values 0.0 and 0.2, between 0.0 and 0.3, 0.0 and 0.4, etc. An advantage of obtaining the second fluorescence signal from the tail of the emission distribution is that the difference between S1 and S2 becomes larger and the division in the equation for P1 becomes numerically more stable since the denominator is closer to 1. However, a disadvantage is that the S2 signal may be considerably more noisy. It may be necessary to increase the integration time, which is not desirable in real-time applications.

Given a specific fluorescence agent and application, a skilled person will be able to determine whether the approach of FIG. 8 or of FIG. 9, both of which correspond to aspects of the invention, is more suitable.

As shown above, by performing calculations based on the first detection signal (S1), the second detection signal (S2) and knowledge of the fluorescence wavelength profile 73, a third detection signal (S3=(S1−S2)/(1−x)) with an improved fluorescence-to-background radiation ratio can be calculated as a function of S1, S2, and x.

Other numerical approaches may also be used. A very simple approach is to simply calculate the difference between the signal S1 at a fluorescence peak and the signal S2 corresponding to a wavelength where the fluorescence profile 73 has a lower fluorescence emission intensity (e.g. at a minimum intensity value in curve 73, or somewhere between the minimum and maximum value), that is S3=S1−S2. In areas where background emission is predominant, the term S2−S1 will mostly cancel, whereas where fluorescence emission is predominant, S2−S1 is positive. In yet another embodiment, the difference between S2 and S1 is normalized, e.g. using S3=(S1−S2)/(S1+S2). In these simplified formulas, the fluorescence profile 73 is not explicitly present. However, the profile 73 is implicitly used, since the wavelength ranges S1 and S2 are chosen based on the known fluorescence profile 73.

Because the light arriving at the sensors follows a single incident light path 42 before being separated in the dichroic prism assembly 52, the detected images of all three sensors are completely aligned. By measuring fluorescence radiation at two separate frequencies for the same spatial location, due to the alignment, the background radiation can be accurately separated from the fluorescence radiation. Moreover, due to the alignment of the three sensors, the separated fluorescence radiation can be accurately superimposed on a visible light (grayscale) image of the surroundings of the endoscope or open lens system.

The detected data will typically be organized in a matrix form with rows and columns to present a digital picture comprising pixels. Each pixel corresponds to a direction of incident radiation. In one of many possible representations, pixels representing a low measured signal are dark and pixels representing a relatively high signal are bright. Based on the determined third detection signal, pixels comprising essentially only background radiation may be darkened. That way, the areas of the image representing fluorescence data will be more clearly visible, and a human operator will be better able to interpret the measurement data.

The shown image may be the third detection signal, or a post-processed (for example, normalized or calibrated) image based on the third detection signal. In an embodiment, the third detection signal is merged with a visible light image, to create an image showing the visible surroundings overlaid with fluorescence data.

As has been shown, from the signals S1 and S2 a ratio of fluorescence to background radiation may be determined. For example, B can be expressed as B=(S2−xS1)/(1−x), so that the fluorescence to background radiation ratio can be expressed as P1/B=(S1−S2)/(S2−xS1). Using such a measure or any other estimate of the fluorescence and background fractions in the signal, there are many ways in which the criterion of “essentially only background radiation” for darkening pixels may be applied. The system can use a hard threshold, for example darkening all pixels with an estimated fluorescence fraction of less than 10% (i.e. 90% background radiation), or less than 20%, or less than 5%. In an alternative embodiment, the pixel is darkened by multiplying its original value with the determined fraction of fluorescence radiation. After such “soft mixing” the image may be re-normalized so that the areas with the most fluorescence radiation have high brightness. In yet another embodiment, the pixel value will be set proportional to the determined fraction of fluorescence radiation.

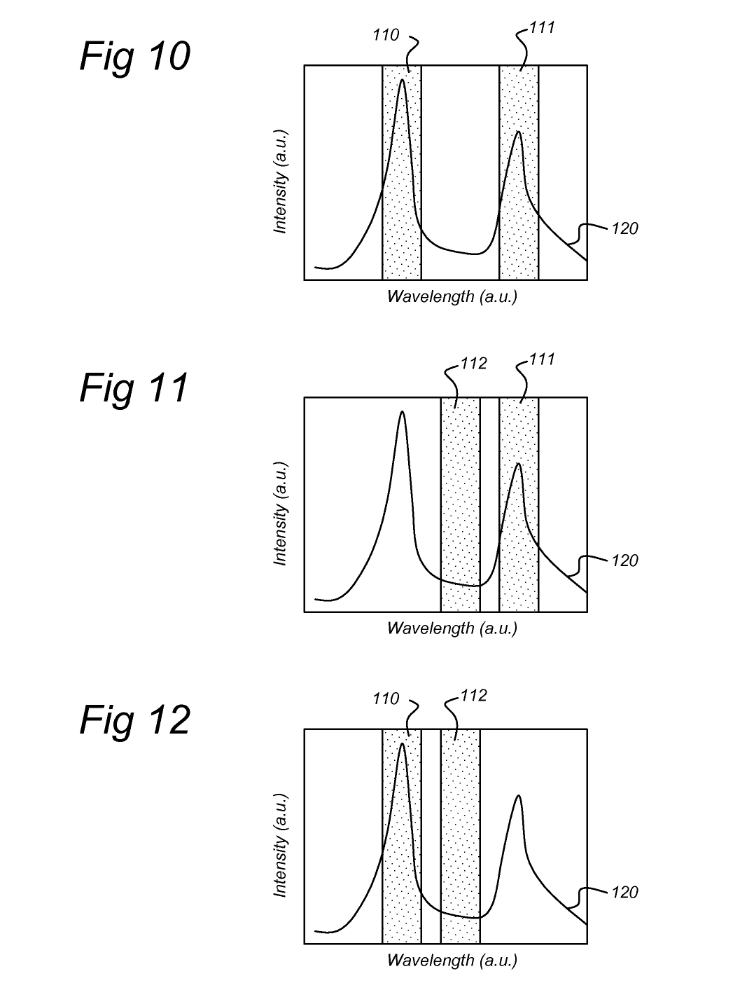

There are fluorescence agents that have multiple peaks in the fluorescence emission distribution. An exemplary distribution 120 having two peaks is schematically shown in FIGS. 10, 11, and 12. According to embodiments of the invention, the first wavelength range 110, 111 can overlap with either emission peak, while the second wavelength range 111, 110, 112 can overlap with either other emission peak (ranges 111, and 110, respectively) or with the “valley” between the peaks (range 112). Using the principles as explained in reference to FIGS. 8 and 9, the skilled person can separate background and fluorescence radiation based on measurements on at least two sampling channels.

While the exemplary embodiments discussed in reference to FIGS. 8 through 12 show only two simultaneous sampling wavelength ranges (74, 81, 91, 110, 111, 112), the invention is not limited to just two simultaneous ranges. Three or more simultaneous ranges may be used. Such increased number of sampling ranges will increase the reliability of the background/fluorescence separation according the invention. In a particular embodiment, a probe is used with a dichroic prism assembly in the tip which has three channels, configured for three fluorescence sampling wavelengths. In yet another embodiment, the probe tip is provided with two dichroic prism assemblies. However, in this embodiment care must be taken to align the measurements from the separate prism assemblies.

The invention has been mainly described in reference to an endoscopy application utilizing an endoscope with a tip as shown in FIGS. 4-6. In particular, the invention can be practised using an endoscope having a tip with integrated miniaturized dichroic prism assembly for wavelength separation. However, the invention may also be applied to other fluorescence probes, such as open systems comprising a lens.

FIG. 13 shows an alternative probe 100 according the invention. The probe 100 has an elongated cylindrical body, comprising main part 101 and distal end or tip 102. The tip 102 is provided with a surface 104 for collecting incident radiation. The incident radiation comprising the fluorescence radiation to be measured will pass through a lens (not shown) in the tip and be collected in a plurality of optical fibers. The fibers will transport the light through the main part 101 of the probe towards a connected analysis unit 105. The analysis unit may comprise a wavelength separation unit, such as a dichroic prism assembly, and sensors with which the invention may be practised. An external light source (not shown) is used to excite the fluorescence agent.

The invention can thus be practiced using endoscopes or other types of probes such as open systems. The light for fluorescence agent excitation may be provided via the system (for example generated in or at least transported through fibers in an endoscope) or external (for example external to an open system probe) The endoscope or probe may comprise wavelength separation means (such as a dichroic prism assembly) at or near the site of incident radiation collection (i.e. in the tip) or in a connected analysis unit to which the incident radiation is transported (for example using optical fibers).

In the foregoing description of the figures, the invention has been described with reference to specific embodiments thereof. It will, however, be evident that various modifications and changes may be made thereto without departing from the scope of the invention as summarized in the attached claims.

In particular, combinations of specific features of various aspects of the invention may be made. An aspect of the invention may be further advantageously enhanced by adding a feature that was described in relation to another aspect of the invention.

It is to be understood that the invention is limited by the annexed claims and its technical equivalents only. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the element is present, unless the context clearly requires that there be one and only one of the elements. The indefinite article “a” or “an” thus usually means “at least one”.

1. Method for detecting fluorescence radiation from a fluorescence agent, the method comprising
emitting light at an excitation wavelength range (72) for causing fluorescence radiation emission in the fluorescence agent, said fluorescence radiation having a fluorescence wavelength profile (73);
detecting light at a first fluorescence wavelength range (74) as a first detection signal (S1);
detecting light at a second fluorescence wavelength range (81, 91) as a second detection signal (S2);
numerically determining a third detection signal with an improved fluorescence-to-background radiation ratio based on the first detection signal (S1), the second detection signal (S2), and the fluorescence wavelength profile (73).

2. Method according to claim 1, further comprising
generating a fluorescence image based on the third detection signal;
showing said fluorescence image on a display.

3. Method according to claim 2, further comprising
detecting visible light as a fourth detection signal;
merging the fluorescence image with an image based on the fourth detection signal.

4. Method according to claim 2, wherein the detected light is captured via a single incident light entry surface, so that the respective detection signals are spatially aligned.

5. Method according to claim 1, wherein numerically determining the third detection signal comprises calculating the difference of the first detection signal (S1) and the second detection signal (S2).

6. Method according to claim 5, wherein numerically determining the third detection signal comprises evaluating (S1−S2)/(1−x), wherein S1 represents a detection signal in the first fluorescence range (74), S2 represents a detection signal in the second fluorescence range (81, 91), and x is the calculated ratio of light emitted in the first florescence wavelength range (74) and light emitted in the second fluorescence wavelength range (81, 91) according to the fluorescence wavelength profile (73).

7. Method according to claim 1, wherein the second fluorescence wavelength range is at a wavelength range (81) where the fluorescence wavelength profile (73) has a normalized value of at least 0.2.

8. Method according to claim 1, wherein the second fluorescence wavelength range is at a wavelength range (91) where the fluorescence wavelength profile (73) has a normalized value that is less than 0.2.

9. Method according to claim 1, wherein the light at the excitation wavelength is emitted from an endoscope tip, and the detectors are comprised in said endoscope tip

10. Method according to claim 1, wherein the light at the excitation wavelength is emitted from a light source external to the probe and the light at the first and/or the second fluorescence wavelength ranges are detected using a prism based camera system.

11. Measurement device for measuring fluorescence radiation from a fluorescence agent having a fluorescence wavelength profile (73), the device comprising
a wavelength separation device (52, 30) configured to receive incident light originating from the agent and to separate said light into a plurality of channels;
at least two imaging sensors connected to at least two respective channels of the plurality of channels, wherein the first channel is configured for transmitting light at a first fluorescence wavelength range (74), from which the respective sensor (14) will generate a first detection signal (S1), and the second channel is configured for light at a second fluorescence wavelength range (81, 91), from which the respective sensor (15) will generate a second detection signal (S2);
a processing device configured for numerically determining a third detection signal with an improved fluorescence-to-background radiation ratio based on the first detection signal (S1), the second detection signal (S2), and the fluorescence wavelength profile (73).

12. The device according to claim 11 configured for use as an endoscope tip, wherein the wavelength separation device is a dichroic prism assembly (52, 30).

13. The device according to claim 12 further provided with fibers (60) for transmitting excitation light to excite the fluorescence agent.

14. Endoscope tip according to claim 12, wherein the dichroic prism assembly (52, 30) has at least three channels, the third channel being configured for transmitting light at a visible wavelength range, from which the respective sensor (16) can generate a fourth signal representative of the visible environment of the endoscope tip.

15. Endoscope system comprising an endoscope tip according to claim 12 and processing means for numerically determining a third detection signal with an improved fluorescence-to-background radiation ratio based on the first detection signal (S1), the second detection signal (S2), and the fluorescence wavelength profile (73).

16. Probe system comprising a device according to claim 11 and processing means for numerically determining a third detection signal with an improved fluorescence-to-background radiation ratio based on the first detection signal (S1), the second detection signal (S2), and the fluorescence wavelength profile (73).

17. Endoscope tip according to claim 13, wherein the dichroic prism assembly (52, 30) has at least three channels, the third channel being configured for transmitting light at a visible wavelength range, from which the respective sensor (16) can generate a fourth signal representative of the visible environment of the endoscope tip.

18. Method according to claim 3, wherein the detected light is captured via a single incident light entry surface, so that the respective detection signals are spatially aligned.
